(19) United States
(12) Patent Application Publication
(10) Pub. No.: US 2012/0100195 A1
(43) Pub. Date: Apr. 26, 2012

(54) DEODORANT OR ANTIPERSPIRANT IMPREGNATED WIPES

(76) Inventors: Marielle Sainz, Miami Beach, FL (US); Irene Heyer, Port Heron, MI (US)

(21) Appl. No.: 13/281,376

(22) Filed: Oct. 25, 2011

Related U.S. Application Data
(60) Provisional application No. 61/406,554, filed on Oct. 25, 2010.

(51) Int. Cl. A61K 8/02; A61K 8/26; A61Q 15/00; A61K 8/28

(52) U.S. Cl. 424/401; 424/66; 424/68

(57) ABSTRACT

An apparatus for facilitating the application of anti-perspirant and/or deodorant to an individual is disclosed. The apparatus comprises a planar element, which may be circular in shape and composed of an absorbent material, a first pocket disposed on a first side of the planar element, wherein the first pocket accommodates the individual's finger, and a deodorant and/or antiperspirant disposed on a second side of the planar element. The apparatus may further include a second pocket disposed on the first side of the planar element adjacent to the first pocket, wherein the second pocket comprises a finger-tip shape, and a wing comprised of a planar element that extends from the first side of the planar element, wherein the wing is connected to the planar element at a location between the first and second pockets. The apparatus may further include an emollient disposed on the second side of the planar element.

### CROSS-REFERENCE TO RELATED APPLICATIONS

This utility patent application claims priority to U.S. provisional patent application No. 61/406,554, filed Oct. 25, 2010 and entitled “Deodorant or Anti-perspirant Impregnated Wipes.” The subject matter of U.S. provisional patent application No. 61/406,554 is hereby incorporated by reference in its entirety.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### FIELD OF THE INVENTION

The invention disclosed broadly relates to the field of cosmetic products, and more particularly relates to the field of deodorant and antiperspirant personal hygiene products.

### BACKGROUND OF THE INVENTION

Deodorants are substances applied to the body to eliminate body odor caused by the bacterial breakdown of perspiration. A subgroup of deodorants, antiperspirants, prevent odor and reduce sweat produced by parts of the body. Antiperspirants are typically applied to the underarms, while deodorants may also be used on feet and other areas in the form of body sprays. Deodorants are classified and regulated as cosmetics by the U.S. Food and Drug Administration (FDA). Deodorants that act as antiperspirants are classified as drugs by the FDA.

During travel or outings, individuals often pack conventional deodorant or antiperspirant bars or sticks for use when perspiring, changing clothing or after bathing. Sticks and bars, however, can be bulky and take up scarce space when packing light or using a small travel package, such as a purse or a pant pocket. It seems unnecessary to carry such a large package of deodorant/antiperspirant when users need only use it once for an overnight event.

Consequently, a need exists to overcome the problems with the prior art as discussed above, and particularly for an applicator of deodorant and antiperspirant that is less bulky and easy to use.

### SUMMARY OF THE INVENTION

Briefly, according to an embodiment of the present invention, an apparatus for facilitating the application of anti-perspirant and/or deodorant to an individual is disclosed. The apparatus comprises a planar element, which may be circular in shape and composed of an absorbent material, a first pocket disposed on a first side of the planar element, wherein the first pocket accommodates the individual's finger, and a deodorant and/or antiperspirant disposed on a second side of the planar element. The apparatus may further include a second pocket disposed on the first side of the planar element adjacent to the first pocket, wherein the second pocket comprises a finger-tip shape, and a wing comprised of a planar element that extends from the first side of the planar element, wherein the wing is connected to the planar element at a location between the first and second pockets. The apparatus may further include an emollient disposed on the second side of the planar element.

The foregoing and other features and advantages of the present invention will be apparent from the following more particular description of the preferred embodiments of the invention, as illustrated in the accompanying drawings.

### BRIEF DESCRIPTION OF THE DRAWINGS

The subject matter, which is regarded as the invention, is particularly pointed out and distinctly claimed in the claims at the conclusion of the specification. The foregoing and other features and also the advantages of the invention will be apparent from the following detailed description taken in conjunction with the accompanying drawings. Additionally, the left-most digit of a reference number identifies the drawing in which the reference number first appears.

FIG. 1 is an illustration of a frontal view of the single use deodorant/antiperspirant pad apparatus according to one embodiment of the present invention.

FIG. 2 is an illustration of a rear view of the single use deodorant/antiperspirant pad apparatus of FIG. 1.

FIG. 3 is an illustration of a perspective view of the single use deodorant/antiperspirant pad apparatus of FIG. 1 shown in a disassembled state.

### DETAILED DESCRIPTION

The present invention provides a single-use, disposable cosmetic wipe that is easy to carry and allows for quick application of the cosmetic (such as a deodorant or antiperspirant) without soiling the user's hands. The present invention improves over the prior art by providing a single-use cosmetic applicator that is thin (one to two millimeters, for example) and occupies a diminutive amount of space similar to a credit card, thereby allowing for easy travel and packing for same. The present invention is designed to be easily transportable and then disposed of after one use. The present invention also allows for cleanly use as the cosmetic is only located on one side of the pad, while the clean side of the pad provides fingertip pockets that allows for ease of application. Further, the present invention allows for worry-free travelling with the apparatus since it is located within an impermeable disposable package that does not allow the cosmetic to escape until it is opened for use. Finally, the present invention provides for a color indicator that indicates when the cosmetic on the apparatus has been depleted and should be disposed of.

The term “cosmetic” as used herein includes substances used to enhance the appearance or odor of the human body. Cosmetics include skin-care creams, lotions, powders, perfumes, lipsticks, fingernail and toe nail polish, eye and facial makeup, hair colors, hair sprays and gels, deodorant, anti-perspirant, hand sanitizer, oils, salts, butters and many other types of products. The term “deodorant” includes substances applied to the body to affect body odor caused by bacterial growth and the smell associated with bacterial breakdown of perspiration in armpits, feet and other areas of the body. Deodorant includes perfumes, fragrances, scents, alcohol, potassium alum, ammonium alum, and the like. The term “anti-perspirant” refers to substances used to quell perspiration in an individual and includes aluminium chloride, aluminium chlorohydrate, aluminium-zirconium compounds, aluminium zirconium tetrachlorohydrex gly, aluminium zirconium trichlorohydrex gly, silica, PEG-8 distearate, aluminum sulfate, metal halides, and the like.

The term “emollient,” also known as a moisturizer, refers to a complex mixture of chemical agents specially designed to make the external layers of the skin (epidermis) softer and more pliable, by increasing its hydration (water content) by reducing evaporation. Naturally occurring skin lipids and sterols as well as artificial or natural oils, humectants, emollients, lubricants, etc. may be part of the composition of commercial skin moisturizers. The terms “pad,” “wipe,” “nap,” “towel,” “towelette,” or “applicator” are used interchangeably to refer to any species of small pieces of paper, cloth, cotton, sponge, foam, or the like, that may come individually wrapped for convenience and are used to apply a substance, such as a cosmetic, to an individual's body.

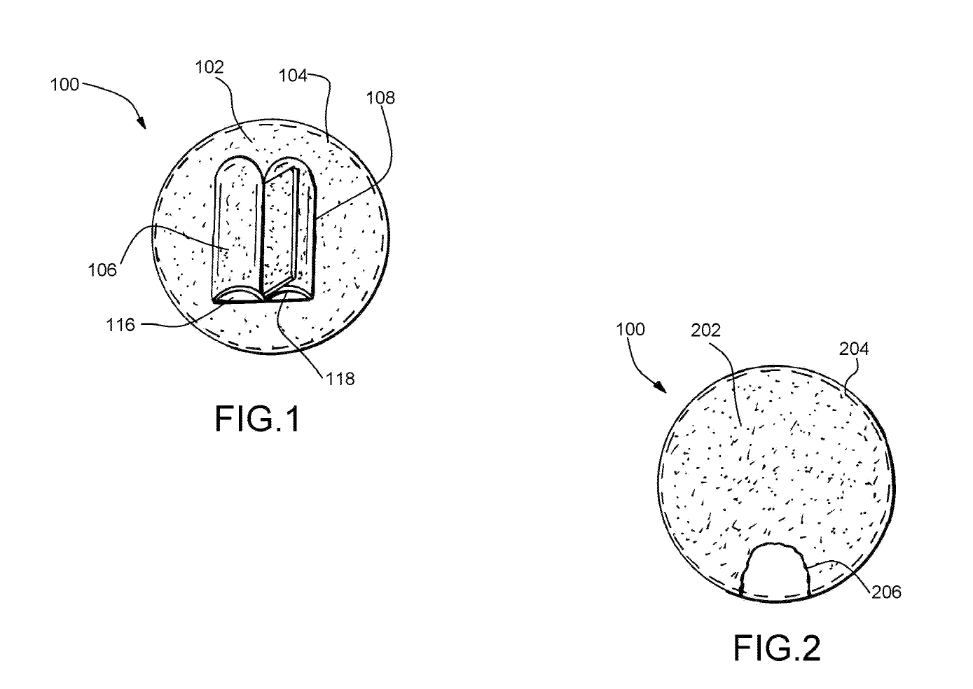

FIG. 1 is an illustration of a frontal view of the single use deodorant/antiperspirant pad apparatus 100 according to one embodiment of the present invention. Apparatus 100 comprises a first planar element 102 having a circular shape. Apparatus 100 also includes a first pocket 106 disposed on a first, or front side of the planar element 102, wherein the first pocket comprises a finger-tip shape, and a second pocket 108 disposed on the front side of the planar element 102 adjacent to the first pocket 106, wherein the second pocket 108 also comprises a finger-tip shape. The cavities 116, 118 formed by the first and second pockets 106, 108 accommodate the insertion of two fingers, such as a user's index finger and fore finger, so that the user may easily use his hand and fingers to manipulate the apparatus 100 and apply the cosmetic to the user's body, such as the axilla or armpit, while keeping the user's hands and fingers away from the cosmetic on the apparatus 100.

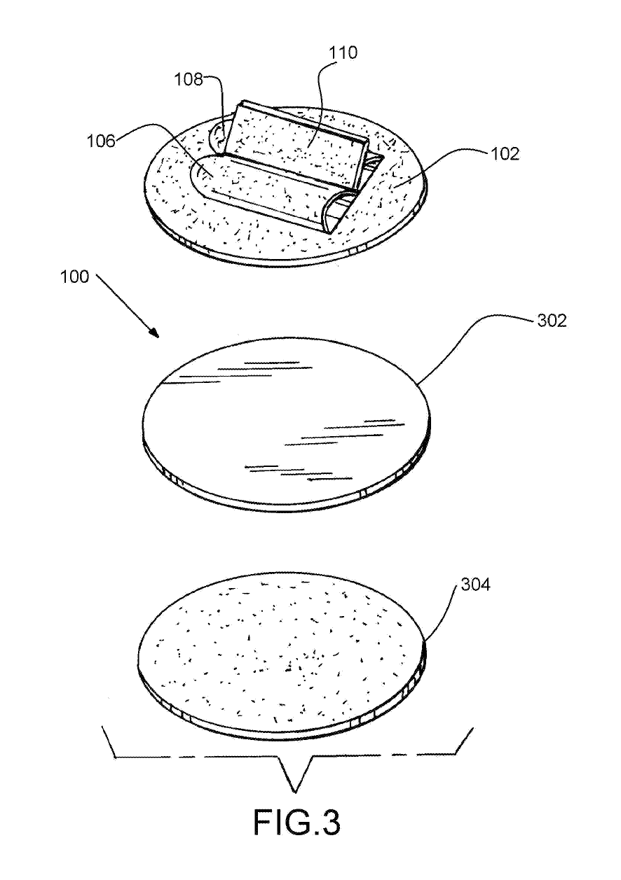

FIG. 1 also include a wing 110 comprised of a planar element that extends from the front side of the planar element 102, wherein the wing 110 is connected to the planar element 102 at a location between the first and second pockets 106, 108. The purpose of the wing 110 is to allow the user to hold the apparatus 100 while the user inserts or removes his fingers from the first and second pockets 106, 108 while eliminating the possibility that the cosmetic on the apparatus soils the user's hands and fingers. FIG. 1 also shows stitching or embossing 104, which serves to connect or couple one or more plies or layers of material, as shown in FIG. 3 below.

Planar elements 102 and 304 may be composed of a soft, absorbent material, such as cotton, a synthetic substitute, or a cotton blend, that allows a cosmetic, such as anti-perspirant or deodorant, to be disposed on its face. The material of planar elements 102 and 304 may also exhibit a characteristic that holds the cosmetic on its face, while also allowing the cosmetic to be divorced of the planar elements, so as to allow a user to wipe the apparatus 100 on his body, such as the axial or armpit, and apply the cosmetic onto the user's body. Planar elements 102 and 304 may be composed of non-woven material such as cotton, or a cotton blend, or of a woven material, such as cotton fabric. Additional materials include paper, felt, foam, sponge, etc. The material of planar elements 102 and 304 may also exhibit additional characteristics, such as being porous, absorbent, elastic, water repellent, moisture wicking, etc. Note the proportion of the size of the pockets 106, 108, the wing 110 and planar element 102 may vary.

FIG. 2 is an illustration of a rear view of the single use deodorant/antiperspirant pad apparatus 100 of FIG. 1. FIG. 2 shows that the underside of planar element 102 comprises a face 202 that may be composed of the same material as that of the element 102. A layer of a cosmetic, such as a deodorant, anti-perspirant and/or emollient, is disposed on face 202. The cosmetic disposed on face 202 or impregnated on face 202 is applied to the user's body when the user wipes the face 202 on his body.

FIG. 2 also shows stitching or embossing 206, which serves as an indicator of an area of the face 202 that is not impregnated with the cosmetic. The purpose of the stitching or embossing 206 is to provide a visual indicator to the user of an area of the face 202 that does not contain the cosmetic, so that the user may place his opposing finger, such as his thumb, in the area shown by stitching 206. Preferably, the user inserts his fore finger and index finger into the first and second pockets 106, 108 and the user places his thumb in the area shown by stitching 206, so that the user may easily use his fingers to manipulate the apparatus 100 and apply the cosmetic to the user's body, while keeping the user's fingers away from the cosmetic on the apparatus 100. Note the area shown by stitching 206 comprises a substantially semicircular shape.

FIG. 3 is an illustration of a perspective view of the single use deodorant/antiperspirant pad apparatus 100 of FIG. 1, shown in a disassembled state. FIG. 3 shows the planar element 102 as the top layer in the apparatus 100. FIG. 3 also shows layer 302 as the middle layer of the apparatus 100. The middle layer 302 may be composed of an impermeable plastic material, such as polyethylene or some similar plastic film. FIG. 3 also shows layer 304 as the bottom layer of the apparatus 100, wherein the outward facing side of the layer 304 is the face 202 of FIG. 2. The impermeable nature of middle layer 302 is such that it does not allow the cosmetic on the face 202 to travel from the bottom layer 304 to the top layer 102, thereby keeping the user's fingers away from the cosmetic on the apparatus 100. Even though the FIG. 3 shows the layers 102, 302, 304 having a circular shape, the present invention supports different shapes such as a square, rectangle, oval, triangle, etc. Recall FIG. 1 shows stitching or embossing 104, which serves to connect or couple the layers of material 102, 302, 306, as shown in FIG. 3. The layers of material 102, 302, 306 may also be coupled using a thermal ultrasonic bonding process.

In an embodiment of the present invention, the single use deodorant/antiperspirant pad apparatus 100 is situated within a disposable package. The disposable package may have a rectangular shape and may be composed of an impermeable plastic material, which completely encompasses the planar element. The impermeable plastic material may comprise polyethylene. The impermeable plastic material may be lined with a foil so that it may withstand long periods of time until it is needed for use. The foil aids in keeping the product moist and protect it from enduring long periods of time without use.

The cosmetic applied to the face 202 of apparatus 100 may be in a solid, liquid, powder or gel form. The cosmetic applied to the face 202 may be an aqueous solution or composition that contains deodorant, anti-perspirant, emollient, fragrance, and/or any other substances that usually accompany aforesaid elements, such as alcohol. The cosmetic applied to the face 202, or any component thereof, may be water soluble for ease of consumer use. The presence of an emollient or moisturizer in the cosmetic applied to the face 202 assists in gliding when the apparatus 100 is applied to the user's skin and provides skin-smoothing aesthetics to the user. The emollient may comprise dimethicone, petrolatum, glycerin or other glycol to aid the user's skin in absorbing moisture and to aid in releasing anti-perspirant from the face 202. The cosmetic applied to the face 202 may also include botanical, which include natural deodorants/antiperspirants and fragrances such as cucumber, lemongrass, green tea, aloe, etc. The cosmetic applied to the face 202 may further include a colorant used for aesthetic purposes and/or to indicate the presence of the cosmetic.

In an embodiment of the present invention, the elements of apparatus 100, such as planar elements 102 and 304 may be impregnated with a dye that adds color to the apparatus 100 for aesthetic and/or functional purposes. The color may be used an indicator as to how much of the cosmetic remains on the face 202. As the cosmetic is depleted, the underlying color of the apparatus 100 may show, thereby indicating the apparatus 100 should be disposed. In another embodiment of the present invention, face 202 of apparatus 100 may include a color-changing strip that changes color as the amount of the cosmetic remaining on the face 202 is depleted. In this embodiment, the color-changing strip may include a litmus-paper-like strip of paper or another material that holds one color when the amount of cosmetic present on the strip is at full capacity. As the cosmetic is depleted, the color of the strip may change, thereby indicating the apparatus 100 should be disposed.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the present invention.

We claim:

1. An apparatus for facilitating the application of anti-perspirant and/or deodorant to an individual, comprising:
a first planar element;
a first pocket disposed on a first side of the first planar element, wherein the first pocket accommodates the individual's finger; and
a deodorant and/or antiperspirant disposed on a second side of the first planar element.

2. The apparatus of claim 1, wherein the first planar element is composed of an absorbent material.

3. The apparatus of claim 2, wherein the first planar element comprises a substantially circular shape.

4. The apparatus of claim 3, wherein the first pocket substantially comprises a finger-tip shape.

5. The apparatus of claim 4, a second pocket disposed on the first side of the first planar element adjacent to the first pocket, wherein the second pocket accommodates the individual's finger.

6. The apparatus of claim 5, wherein the second pocket substantially comprises a finger-tip shape.

7. The apparatus of claim 6, further comprising a wing comprised of a planar element that extends from the first side of the first planar element, wherein the wing is connected to the first planar element at a location between the first and second pockets.

8. The apparatus of claim 7, wherein the deodorant and/or antiperspirant disposed on the second side of the first planar element comprises a water-soluble anti-perspirant.

9. The apparatus of claim 7, wherein the deodorant and/or antiperspirant disposed on the second side of the first planar element comprises any one of aluminium zirconium tetrachlorohydrex gly and aluminium chlorohydrate.

10. The apparatus of claim 7, further comprising an emollient disposed on the second side of the first planar element.

11. The apparatus of claim 1, further comprising a second planar element attached to the second side of the first planar element, wherein the second planar element is composed of a non-permeable material.

12. The apparatus of claim 11, further comprising a third planar element attached to an outward facing side of the second planar element, wherein the third planar element is composed of an absorbent material, and wherein the deodorant and/or antiperspirant is disposed on an outward facing side of the third planar element.

13. The apparatus of claim 10, further comprising a visual indicator on the second side of the first planar element that indicates an area of the second side of the first planar element that lacks any deodorant and/or antiperspirant, wherein the indicator has a substantially semicircle shape.

14. The apparatus of claim 13, further comprising a dye adding color to the first planar element.

15. The apparatus of claim 14, further comprising a color-changing strip disposed on the second side of the first planar element that indicates when the deodorant and/or antiperspirant is depleted.

16. An apparatus for facilitating the application of a cosmetic to an individual, comprising:
a planar element having a substantially circular shape, wherein the planar element is composed of an absorbent material;
a first pocket disposed on a first side of the planar element, wherein the first pocket comprises a finger-tip shape;
a second pocket disposed on the first side of the planar element adjacent to the first pocket, wherein the second pocket comprises a finger-tip shape;
a cosmetic disposed on a second side of the planar element.

17. The apparatus of claim 16, further comprising a wing comprised of a planar element that extends from the first side of the planar element, wherein the wing is connected to the planar element at a location between the first and second pockets.

18. The apparatus of claim 17, further comprising a visual indicator on the second side of the planar element that indicates an area of the second side of the planar element that lacks any deodorant and/or antiperspirant, wherein the indicator has a substantially semicircle shape.

19. The apparatus of claim 18, further comprising a color-changing strip disposed on the second side of the planar element that indicates when the cosmetic is depleted.

20. An apparatus for facilitating the application of a cosmetic to an individual, comprising:
a planar element having a substantially circular shape, wherein the planar element is composed of an absorbent material;
a first pocket disposed on a first side of the planar element, wherein the first pocket comprises a finger-tip shape;
a second pocket disposed on the first side of the planar element adjacent to the first pocket, wherein the second pocket comprises a finger-tip shape;
a cosmetic disposed on a second side of the planar element, wherein the cosmetic includes one or more of a deodorant, an anti-perspirant and an emollient;
a wing comprised of a planar element that extends from the first side of the planar element, wherein the wing is connected to the planar element at a location between the first and second pockets; and
a rectangular package composed of an impermeable plastic material, which completely encompasses the planar element.
